(19) United States
(12) Patent Application Publication
(10) Pub. No.: US 2008/0177380 A1
(43) Pub. Date: Jul. 24, 2008

(54) Methods and devices for heart tissue repair

(76) Inventors: Niel F. Starksen, Los Altos Hills, CA (US); Mariel Fabro, Mountain View, CA (US); Karl S. Im, Palo Alto, CA (US); Tenny C. Calhoun, Mountain View, CA (US)

Correspondence Address: MORRISON & FOERSTER LLP, 755 PAGE MILL RD, PALO ALTO, CA, 94304-1018, US

(21) Appl. No.: 11/656,141

(22) Filed: Jan. 19, 2007

(51) Int. Cl. A61F 2/24

(52) U.S. Cl. 623/2.11

(57) ABSTRACT

Methods for securing implants to heart tissue are described, where the implants include one or more anchor portions extending from a support. Some variations of the methods may comprise securing an implant to a first region of a tissue in the vicinity of a subannular groove of a heart, where the implant comprises a support and a first anchor portion extending from a first portion of the support. The implant may further comprise a second anchor portion extending from a second portion of the support. Certain variations of the methods may include advancing a catheter to a first region of a tissue in the vicinity of a subannular groove of a heart, and deploying an implant from the catheter to the first region of tissue, where the implant comprises a support and a first anchor portion extending from a first portion of the support. Implants also are described.

### TECHNICAL FIELD

The methods and devices described herein relate generally to the field of implants for heart tissue repair. More specifically, the methods and devices described here relate to implants for heart tissue, where the implants include one or more anchor portions extending from a support. The methods and devices described herein may have particular utility in the area of mitral valve repair.

### BACKGROUND

Advances have been made in the techniques and tools used in minimally invasive heart surgery. As an example, anchors have been developed for use in mitral valve repair. In some mitral valve repair procedures, anchors are deployed into a region of mitral valve tissue. The anchors are secured into the tissue, and also are joined to each other by a tether that is fixedly coupled to one anchor, and slidably coupled to the other anchors. After the anchors have been secured into the tissue, the tether is pulled proximally, thereby reducing the distance between the anchors and reshaping the mitral valve annulus.

In a heart surgery procedure, it would be desirable to minimize procedure time by, for example, minimizing the number of individual deliveries and deployments of devices and anchors to a target site. At the same time, in a mitral valve repair procedure utilizing anchors, it would be desirable to deliver a sufficient number of anchors to the mitral valve tissue to successfully perform the repair procedure. Accordingly, additional methods for efficiently delivering multiple anchors to a target heart tissue (e.g., a mitral valve tissue) would be desirable. Furthermore, delivery devices configured to efficiently deliver devices and anchors to a target heart tissue would also be desirable.

### BRIEF SUMMARY

Described here are methods and devices for heart tissue repair. In general, the methods comprise securing implants to heart tissue (e.g., mitral valve tissue), where the implants include one or more anchor portions extending from a support. Some of the methods described here generally comprise securing an implant to a first region of a tissue in the vicinity of a subannular groove of a heart, where the implant comprises a support and a first anchor portion extending from a first portion of the support. The implant may be secured to tissue by, for example, deploying the implant into the tissue, and allowing the first anchor portion to self-secure into the tissue. In some variations, the implant may further comprise a second anchor portion extending from a second portion of the support. The methods may also comprise securing the second anchor portion into a second region of heart tissue. The second anchor portion may be secured into the second region of heart tissue by deploying the implant to the second region of heart tissue, and allowing the second anchor portion to self-secure into the second region. Certain variations of the methods described here may include tensioning the support prior to securing the implant to a region of heart tissue.

Implants also are described here. Some of the implants generally comprise a support and a plurality of anchor portions extending from the support, where the plurality of anchor portions comprise at least three anchor portions having a non-linear relationship with respect to each other, and the implant is configured to secure to a first region of heart tissue, such as mitral valve tissue. Certain of the implants generally comprise a support and a plurality of anchor portions extending from the support, where the plurality of anchor portions form a non-linear array on the support, and the implant is configured to secure to a first region of heart tissue, such as mitral valve tissue. The anchor portions may be any suitable anchor portions capable of securing into heart tissue, including but not limited to T-tags, rivets, staples, hooks, spikes, anchors, barbs, and clips. In some variations, the anchor portions comprise a plurality of hooks. The implants may have any number of anchor portions. In certain variations, disengagement of at least one of the anchor portions from the heart tissue is unlikely to result in disengagement of the implants themselves from the heart tissue (e.g., because other anchor portions may remain secured to the heart tissue). In this way, anchor portion redundancy is achieved. Anchor portions may be integrally formed with the support or may be attached to the support (e.g., partially embedded in the support). The implants may include both anchor portions that are integrally formed with the support and anchor portions that are attached to the support. Furthermore, the implants may include anchor portions having different sizes and/or shapes.

The support may be formed of one or more polymers, metals, and/or metal alloys, and may be formed of the same material as the anchor portions, or of a different material from the anchor portions. In some variations, the support may be in the form of a generally cylindrical member. The generally cylindrical member may include at least one lumen. The support may include two surfaces that are different from each other (e.g., that are opposite each other), and at least one anchor portion may extend from each surface. The support may include multiple portions. Two different portions of the support may be connected to each other by a spring or by a third portion of the support. The third portion may be integrally formed with one or both of the other two portions.

In some variations, the implants further comprise a tether, such as a suture. The tether may be formed of one or more polymers (e.g., polyester impregnated with polytetrafluoroethylene), and may or may not be attached to the support. In certain variations, the tether is attached to the support. For example, the tether may be attached to one end portion of the support, but not to another end portion of the support. In variations where a tether is used, the methods may include pulling the tether proximally. In some variations, the tether may be pulled proximally to create at least one heart tissue fold between the first and second anchor portions. In certain variations, pulling the tether proximally may result in a reduction of the circumference of the mitral valve annulus.

Other methods described here comprise advancing a catheter to a first region of a heart tissue, and deploying an implant from the catheter (e.g., by using a pusher to advance the implant through the catheter) to a first region of heart tissue, where the implant comprises a support and a first anchor portion extending from a first portion of the support. The catheter typically includes a lumen, and in some variations, the implant is at least partially disposed within the lumen of the catheter (e.g., in a rolled or folded configuration) prior to deployment of the implant to the first region of heart tissue. As the implant is deployed from the catheter, the implant may unroll or unfold, and become secured to the target tissue. In some variations, the catheter may include an expandable member, such as a balloon, that may be used to deploy the implant to the heart tissue. In certain variations, the expandable member may be formed of a shape-memory material. Prior to deployment, the implant may be supported by the expandable member, and/or may be disposed between a sheath and the expandable member. The expandable member may be expanded to deploy the implant to the first region of the heart tissue. In certain variations, deploying the implant from the catheter may include withdrawing the sheath.

### BRIEF DESCRIPTION OF THE DRAWINGS

FIG. 1 is a cross-sectional view of a heart.

FIG. 2 is a cross-sectional view of a portion of a heart.

FIGS. 3A-3G provide a detailed depiction of a method for advancing a sheath to a subannular groove of a heart to deploy an implant to a region of the heart valve annulus.

FIG. 4A is a perspective view of an implant, and FIG. 4B is a side view of the implant of FIG. 4A, secured to tissue. FIG. 4C is a side view in partial cross-section of the implant of FIGS. 4A and 4B disposed within a lumen of a delivery sheath. FIG. 4D depicts the deployment of the implant of FIGS. 4A-4C from the delivery sheath.

FIGS. 5A-5C depict the delivery and deployment of an implant to the tissue of a subject.

FIGS. 6-9 are perspective views of illustrative implants.

FIG. 10 is a side view of an illustrative implant.

FIGS. 11-19 are perspective views of illustrative implants.

FIGS. 20A-20C depict the delivery and deployment of an implant to the tissue of a subject.

FIGS. 21A and 21B depict the delivery and deployment of an implant to the tissue of a subject. FIG. 21C is a side view of the implant of FIGS. 21A and 21B, disposed within a lumen of a delivery sheath.

FIG. 22A is a side view of an implant secured to the tissue of a subject, and FIG. 22B depicts the implant of FIG. 22A forming folds in the tissue of the subject.

FIGS. 23A and 23B depict the delivery and deployment of an implant to the tissue of a subject. FIG. 23C is a side view of the implant of FIGS. 23A and 23B, secured to the tissue of the subject.

FIG. 24A is a side view of an illustrative implant.

FIG. 24B is a side view of the implant of FIG. 24A, as the implant is being adjusted for deployment to a tissue of a subject.

FIG. 24C is a side view of the implant of FIGS. 24A and 24B, secured to the tissue of a subject.

FIG. 25A is a side view of an illustrative implant.

FIG. 25B is a side view of the implant of FIG. 25A, secured to the tissue of a subject.

FIG. 26A is a perspective view of an illustrative implant.

FIG. 26B is a perspective view of the illustrative implant of FIG. 26A, secured to the tissue of a subject.

FIGS. 27-29 are perspective views of illustrative implants.

FIGS. 30-31 are partial cross-sectional views of illustrative implants.

### DETAILED DESCRIPTION

Described here are methods for delivering and securing implants to heart tissue of a subject, such as mitral valve tissue, where the implants comprise a support and one or more anchor portions extending from the support. Variations of the implants also are described. The anchor portions may be any suitable or desirable anchor portions, and may be integrally formed with the support or attached to the support. An implant may include any number of anchor portions, which may or may not be the same size and/or shape.

In some variations, the implants are secured to tissue in the region of the mitral valve. An implant may include a relatively large number of anchor portions extending from a support. In this way, the implant may have a reduced likelihood of unsecuring from the tissue in the event that one anchor portion fails or becomes disengaged from the tissue. In certain variations, the implants may include a support that is capable of being stretched or tensioned during the tissue securing process. Upon release of the support, these implants may create one or more tissue folds. Alternatively or additionally, an implant may include a tether that can be pulled proximally to create one or more folds in the tissue to which the implant is secured.

Heart tissue repair procedures using the implants described here may be relatively efficient and effective. For example, some variations of the methods described here may include deploying only one implant into heart tissue, and using the one implant to form one or more folds in the heart tissue. The result may be that procedure time is reduced relative to methods that include deploying multiple individual implants and/or anchors into heart tissue to form folds in the heart tissue.

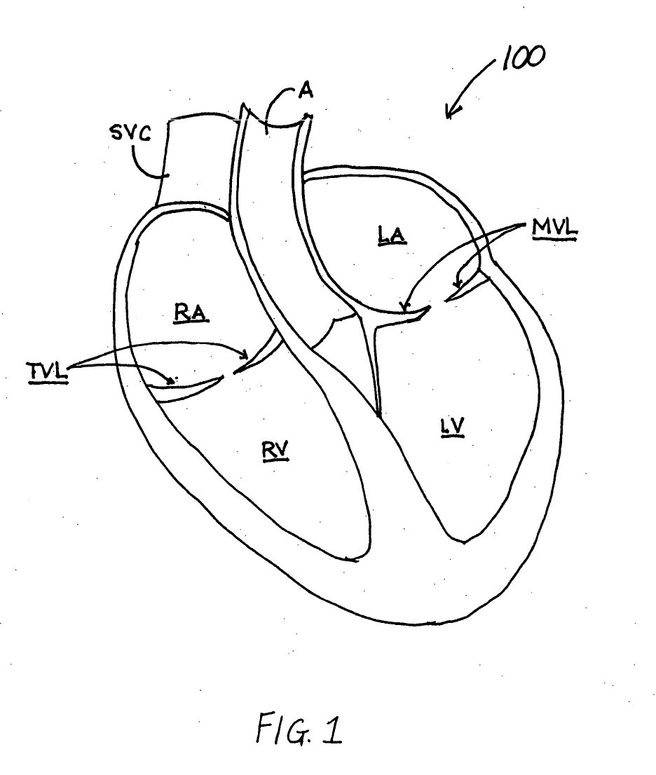

Turning now to the figures, FIG. 1 is a cross-sectional schematic representation of a heart (100). As shown, heart (100) includes the superior vena cava (SVC), the right atrium (RA), the right ventricle (RV), the tricuspid valve leaflets (TVL), the aorta (A), the mitral valve leaflets (MVL), the left atrium (LA), and the left ventricle (LV). The tricuspid valve separates the right atrium (RA) from the right ventricle (RV), and the mitral valve separates the left atrium (LA) from the left ventricle (LV). The mitral valve has two leaflets (MVL), the anteromedial leaflet and the posterolateral leaflet. Surrounding the opening of the mitral valve is a fibrous ring known as the mitral valve annulus. A normally functioning mitral valve allows blood to flow into the left ventricle during ventricular diastole, and prevents blood from going from the ventricle to the left atrium during systole, in a retrograde fashion. A mitral valve that allows blood to flow into the left atrium is said to have regurgitation, and in instances where the regurgitation is severe, mitral valve repair may be desirable.

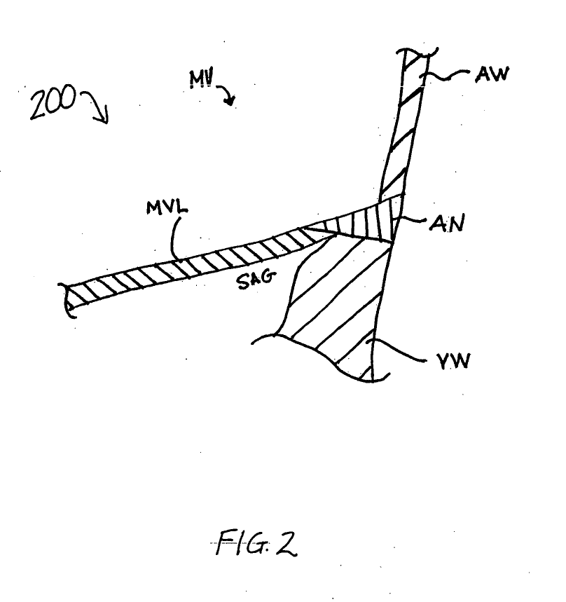

FIG. 2 provides a schematic cross-sectional view of a portion 200 of the mitral valve (MV) anatomy. As shown, portion (200) includes a mitral valve leaflet (MVL). The annulus (AN), which surrounds the valve, is also shown. As FIG. 2 shows, the subannular groove (SAG) is the track defined by the joinder of the horizontal underside of the mitral valve annulus (AN) with the ventricular wall (VW). An equivalent subannular groove is positioned on the underside of the tricuspid valve, and the methods and devices described here may be used with respect to the tricuspid valve as well.

Any suitable method of accessing the SAG may be used. For example, in a retrograde arterial access approach, a catheter or sheath may be inserted into a femoral artery and passed from the femoral artery into the left ventricle, via the aorta (A) and the aortic valve. In an interatrial septal approach, a catheter or sheath may be inserted into a femoral vein and passed from the femoral vein into the right atrium (RA), and may then be advanced from the right atrium into the left atrium via the foramen ovale, and subsequently past the mitral valve and into the left ventricle. Once in the left ventricle, the distal portion of the catheter or sheath, upon further advancement, will naturally travel under the posterolateral valve leaflet into the SAG. The catheter or sheath may be further advanced along the SAG, either partially or completely around the circumference of the valve. It is often desirable to have the catheter or sheath seated at the intersection of the mitral valve leaflets (MVL) and the ventricular wall, adjacent to, and very near the annulus from the underside. The use of a pre-shaped catheter or sheath (e.g., a catheter or sheath having a pre-shaped distal end or portion) may aid in placement by conforming to the target anatomy. While the approach described above employs access through a femoral artery or the femoral vein, access may be obtained through other suitable vessels as well (e.g., the jugular or subclavian artery and vein).

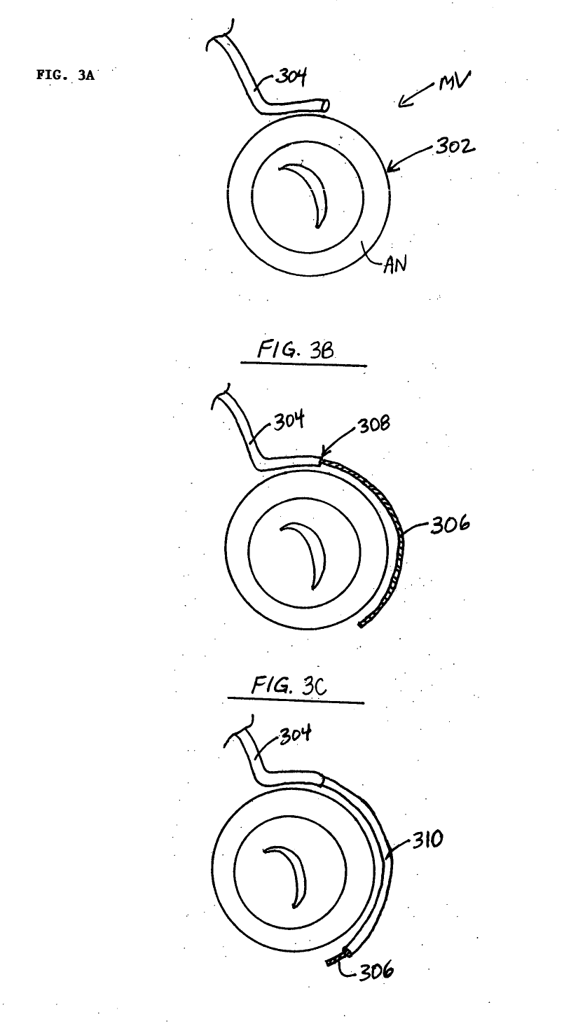

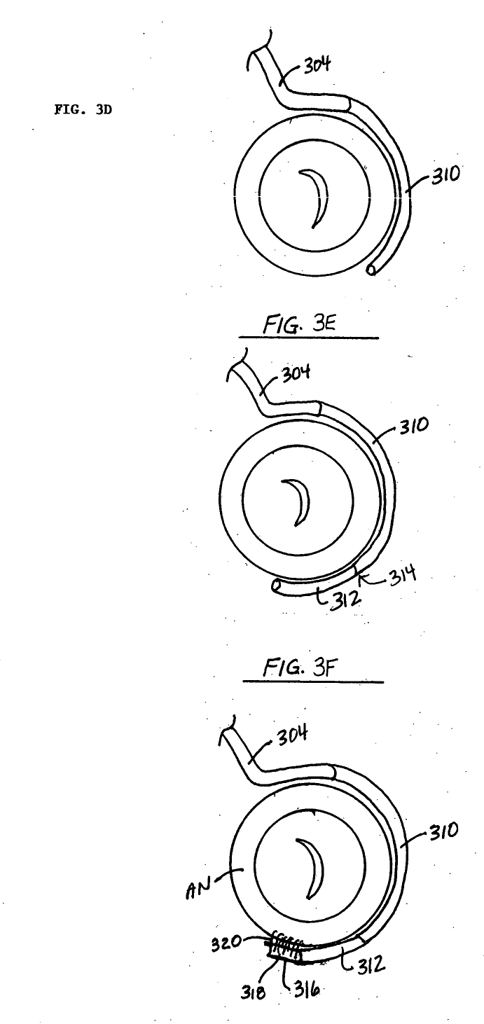

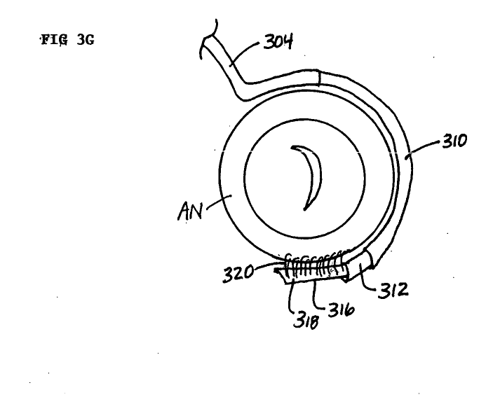

FIGS. 3A-3G provide a detailed depiction of a method for delivering and securing an implant to a region of a heart valve annulus, where the implant includes a support and multiple anchor portions extending from the support. In FIGS. 3A-3G, the mitral valve (MV) of FIG. 2 is depicted schematically from an inferior perspective looking up. Referring to FIG. 3A, a guide catheter (304) is advanced to SAG (302) using any of the access routes (or any other suitable access routes) previously described. After guide catheter (304) has been positioned at the desired location in SAG (302), a guidewire (306) is advanced through the lumen of guide catheter (304). Guidewire (306) is advanced beyond the distal end (308) of guide catheter (304), so that guidewire (306) extends further along SAG (302) than guide catheter (304), as shown in FIG. 3B.

After guidewire (306) has been positioned in the SAG, a tunnel catheter (310) is advanced through guide catheter (304), over guidewire (306), which is shown in FIG. 3C. Tunnel catheter (310) may be any suitable catheter, and in some instances, it is desirable that the tunnel catheter be pre-shaped or pre-formed at its distal end, such as the tunnel catheter illustrated in FIG. 3C. As shown there, the tunnel catheter has a pre-shaped distal portion comprising a curve. In this way, the tunnel catheter may more easily conform to the geometry of the mitral valve. It should also be understood that while one distal curve is shown, any of the catheters or guidewires described here may be pre-shaped or pre-formed to include any number of suitable curves. Of course, the guidewires and/or catheters described here may also be steerable.

After tunnel catheter (310) has been positioned in the SAG, guidewire (306) is withdrawn proximally as shown in FIG. 3D. After guidewire (306) has been withdrawn, a delivery sheath (312) may then be advanced through the lumen of the tunnel catheter (310), and past the distal end (314) of tunnel catheter (310), as shown in FIG. 3E. Next, and as shown in FIG. 3F, delivery sheath (312) is proximally withdrawn as an implant (316) is deployed from delivery sheath (312) into the SAG. Implant (316) includes a support (318) and multiple anchor portions, shown here as hooks (320), extending from the support. The implant may be deployed from the delivery sheath in any suitable fashion. For example, the implant may be deployed using a push-pull wire (e.g., by pushing the wire distally or pulling the wire proximally), using a plunger, or using any other suitable actuation technique. In FIG. 3F, the proximal withdrawal of delivery sheath (312) operates in conjunction with one or more of these actuation techniques to deploy implant (316) into the SAG. However, some variations of the methods described here may not include using both sheath withdrawal and actuation techniques to deploy the implant. For example, certain variations of the method described here may include only using one or more actuation devices, such as a pusher, to deploy the implant, without also withdrawing a sheath.

As shown in FIG. 3F, when implant (316) is deployed into the SAG or tissue in the region of the annulus, hooks (320) engage the tissue in the region of the annulus, thereby securing implant (316) to the tissue. The presence of multiple hooks on the implant can result in the implant being relatively well-secured to the tissue. It should be understood that the hooks may be deployed into the annular tissue directly, or slightly below the annular tissue in the vicinity of the SAG generally.

While the implant shown in FIG. 3F comprises hooks, the anchor portions for use with the methods and devices described here may have any suitable configuration or geometry. Similarly, the anchor portions may be made of any suitable material, and may be any suitable size. In addition, the anchor portions may be made of one material or more than one material. Examples of anchor portion materials include super-elastic or shape memory materials, such as nickel-titanium alloys and spring stainless steel. Examples of suitable anchor portion geometries include T-tags, rivets, staples, hooks (e.g., C-shaped or semicircular hooks, curved hooks of other shapes, straight hooks, barbed hooks), anchors, barbs, and clips. The anchor portions may be configured to self-expand and self-secure into tissue, but need not be configured in such a fashion. Illustrative examples of suitable implants are described in more detail, for example, in U.S. patent application Ser. Nos. 10/461,043, 10/656,797, 10/741,130, 10/776,682, 10/792,681, 10/901,019, 10/901,555, 10/901,554, 10/901,445, 10/901,444, and 11/202,474, all of which are hereby incorporated by reference in their entirety.

After a portion of implant (316) has been deployed in the region of the SAG, delivery sheath (312) is proximally withdrawn by an additional amount, as shown in FIG. 3G, to deploy an additional portion of implant (316) from delivery sheath (312). As the additional portion of the implant is deployed from delivery sheath (312), additional hooks (320) on the implant become exposed and secured into the tissue in the region of the annulus. Eventually, the entirety of implant (316) may be deployed from delivery sheath (312) in this fashion, securing implant (316) to the tissue in the region of the annulus.

Certain variations of the methods described here may include tensioning or stretching support (318) between successive deployments of different portions of implant (316). For example, one portion of implant (316) may be secured to the tissue in the region of the annulus, then support (318) may be tensioned or stretched, and another portion of implant (316) may be secured to the tissue in the region of the annulus. Once the entire implant has been deployed and its various portions have been secured to the tissue in the region of the annulus, the support can be released, thereby resuming its original form and creating one or more folds in the tissue. While the above-described method includes tensioning and stretching the support of the implant substantially throughout the entire deployment process, certain variations of the methods described here may include tensioning or stretching and releasing the support only for selected periods of a deployment process.

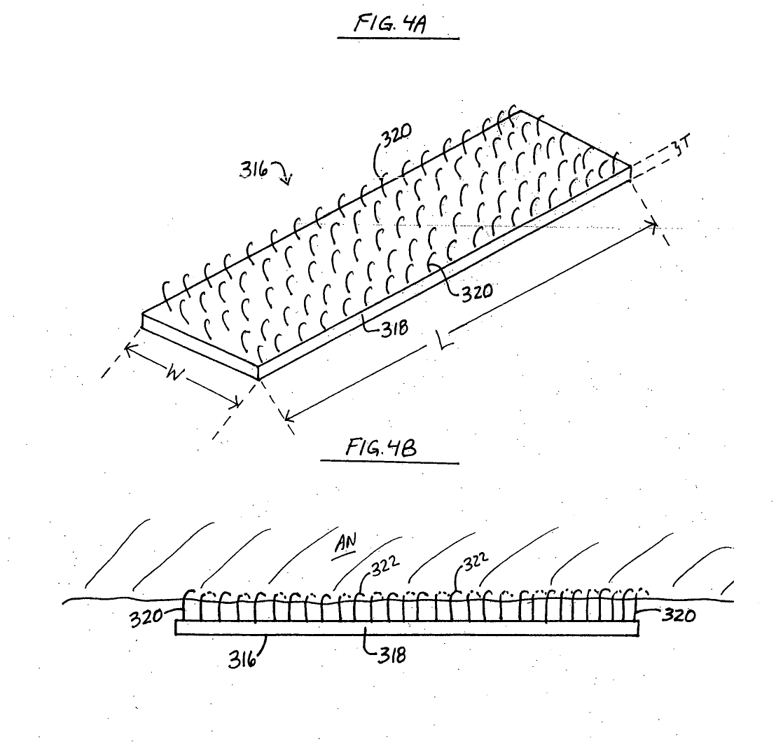

FIG. 4A shows implant (316) in greater detail. As shown in FIG. 4A, implant (316) includes support (318) and anchor portions, shown here as hooks (320), extending from support (318). Support (318) has a length (L), a width (W), and a thickness (T). One or more of these dimensions of support (318) may be selected, for example, based on the dimensions of the site to which implant (316) is to be secured, and/or the dimensions of the delivery device that is used to deploy implant (316) to the target site. In some variations, length (L) can be from about 30 millimeters to about 70 millimeters, width (W) can be from about 2 millimeters to about 3 millimeters, and/or thickness (T) can be from about 0.5 millimeter to about 1 millimeter.

Support (318) may be made from any suitable biocompatible material. For example, the support may be made from one or more polymers. Examples of polymers include polyether-block co-polyamide polymers, copolyester elastomers, thermoplastic polyester elastomers, thermoplastic polyurethane elastomers, polyolefins (e.g., polyethylene, polypropylene), polyurethanes, polystyrenes, polycarbonates, polyesters, polyamides, polyetheretherketones (PEEKs), polytetrafluoroethylene or expanded polytetrafluoroethylene, and silicones. Other examples of materials that may be suitable for support (318) include metals, metal alloys, and shape-memory materials (e.g., shape-memory polymers, nickel-titanium alloys, or spring stainless steel). In some variations, support (318) may include one or more biodegradable materials. As described in further detail below, a tissue-securing process may include temporarily tensioning or stretching an implant support. The support may be formed of one or more materials that accommodate this temporary tensioning or stretching, such as one or more shape-memory materials.

Similarly, the anchor portions, shown here as hooks (320), may be made of one material or more than one material. Examples of suitable materials for anchor portions include metals, metal alloys, polymers, and super-elastic or shape-memory materials, such as nickel-titanium alloys and spring stainless steel. In certain variations, hooks (320) may be made of one or more materials that are selected to impart flexibility to hooks (320). This flexibility may, for example, allow hooks (320) to flex to fit within delivery sheath (312).

Hooks (320) may be made of the same material as support (318), or may be made of one or more different materials from support (318). As an example, in some variations, hooks (320) may be made of a nickel-titanium alloy, while support (318) may be made of a polymer. In certain variations, hooks (320) may be formed separately from support (318), and later attached to support (318). In other variations, hooks (320) may be integrally formed with support (318). Furthermore, some variations of implant (316) may include both hooks (320) that are integrally formed with support (318), and hooks (320) that are attached to support (318).

An implant such as implant (316) may be formed using any number of suitable methods. In variations in which one or more of the hooks of the implant are attached to the support, the hooks may be attached to the support by partially embedding the hooks in the support, adhesive-bonding the hooks to the support, and/or welding the hooks to the support. In variations in which one or more of the hooks of the implant are integrally formed with the support, the hooks may be molded from the support material or stamped or cut into the support material, or the support and the integrally formed hooks may be formed using an extrusion process.

FIG. 4B shows implant (316), with hooks (320) secured to the tissue in the region of the annulus (AN). In the variation described here, hooks (320) have curved heads (322) that all face in the same direction. This unity in direction may, for example, result in a reduced likelihood of implant (316) unsecuring from the tissue. Furthermore, once the hooks have been secured to the tissue, the support may be pulled in the direction in which the hooks' heads are facing. This may also result in a reduced likelihood of implant (316) unsecuring from the tissue. It should be understood, however, that some variations of implants may include hooks with heads that face in different directions. The different hooks may, for example, be used to engage different regions of tissue. Hooks with heads facing in different directions are described in further detail below.

FIGS. 4C and 4D show implant (316) being delivered from delivery sheath (312). As shown in FIG. 4C, delivery sheath (312) includes a lumen (410), within which implant (316) is disposed in a partially rolled form. As shown in FIG. 4D, delivery sheath (312) is proximally withdrawn and an actuator, such as a pusher (not shown), is used to deploy implant (316) from delivery sheath (312). As the implant is deployed, it begins to unroll and flatten out. Additionally, hooks (320), which were compressed by the side wall (412) of sheath (312), resume their hook shapes as they exit the restraints of delivery sheath (312).

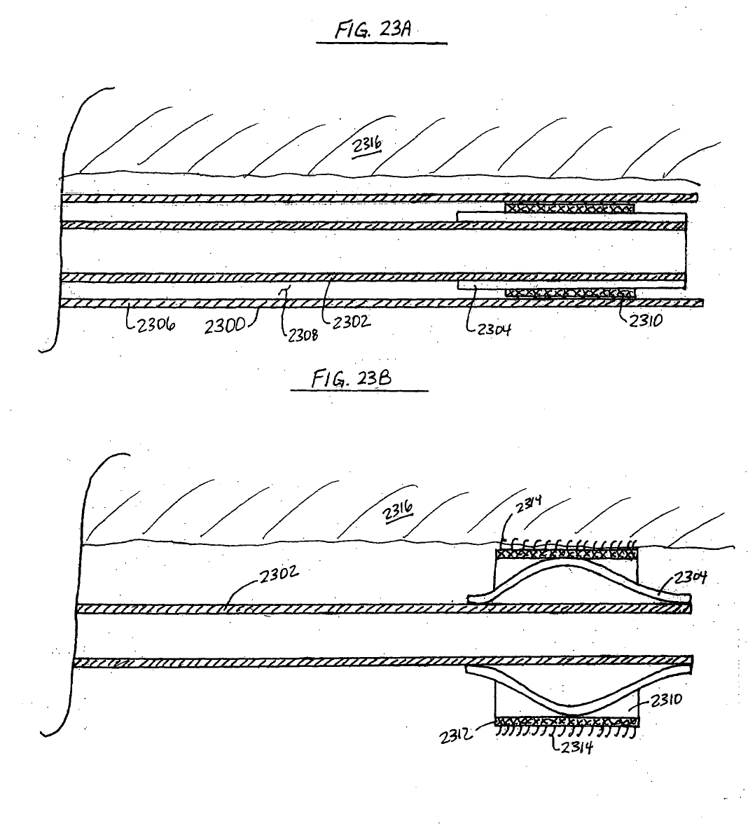

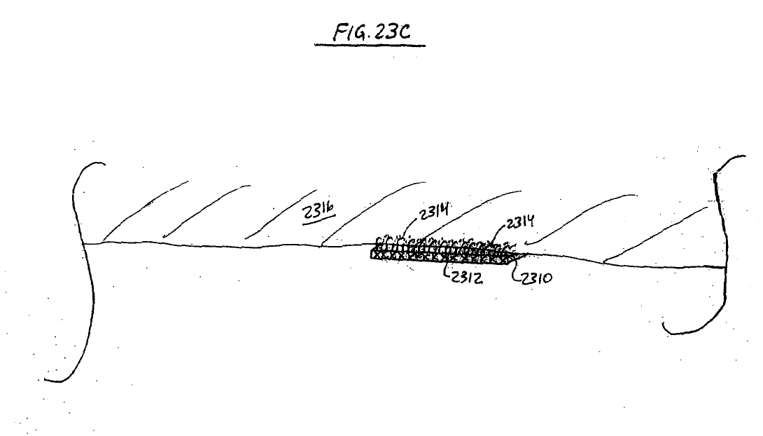

While the above-described methods include using only a delivery sheath to deliver an implant to tissue, other methods may include using one or more additional delivery devices. For example, in some variations, a delivery catheter including an inner member and an outer sheath may be used to deliver an implant to a target tissue. The implant may be disposed within a space between the inner member and the outer sheath, and the delivery catheter may be advanced to a target tissue. Thereafter, the sheath may be retracted, and the implant may be deployed from the delivery catheter and secured to the tissue. As an example, FIG. 23A shows a delivery catheter (2300) including an inner member (2302), an expandable member (2304) disposed on inner member (2302), and an outer sheath (2306) surrounding inner member (2302). As shown, expandable member (2304) is a balloon, but other suitable expandable members may be used. The inner member and outer sheath of the delivery catheter define a lumen (2308) therebetween. An implant (2310) is disposed on expandable member (2304), such that the implant is located within lumen (2308). As shown in FIG. 23B, the implant is deployed when sheath (2306) is proximally withdrawn and expandable member (2304) is expanded. In this way, implant (2310) unrolls and secures to tissue (2316). As with the other implants described here, implant (2310) includes a support (2312) and anchor portions (2314) extending from the support. As implant (2310) is deployed, anchor portions (2314) secure into tissue (2316).

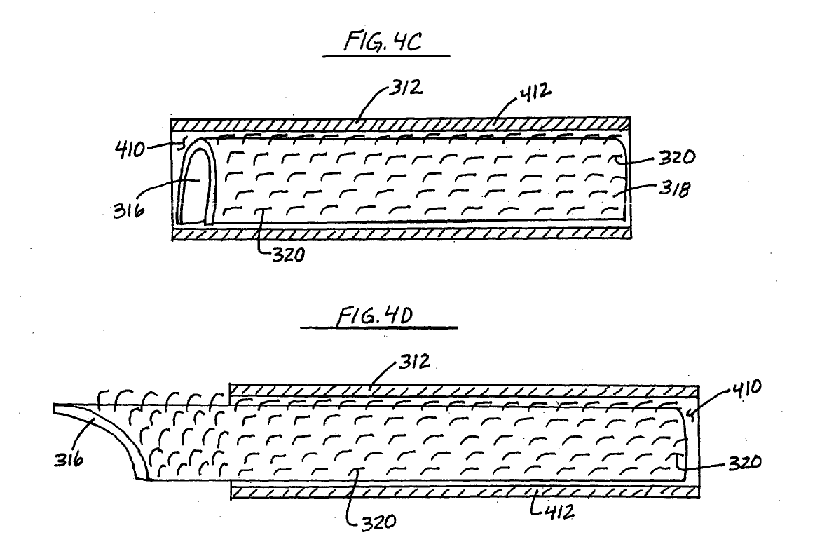

As described above, some methods may include securing one portion of an implant to tissue, stretching a support of the implant, securing another portion of the implant to the tissue, and then releasing the implant to allow it to form one or more folds in the tissue. FIGS. 5A-5C depict one variation of such a method in further detail. FIG. 5A shows a delivery sheath (500) that is used to deliver an implant (502) to a region of tissue (508). The implant may be deployed from the delivery sheath using any of the deployment techniques described above. For example, the implant may be deployed from the sheath by proximally withdrawing the sheath and pushing the implant out of the sheath with a pusher. Implant (502) includes a support (504) from which a hook (506) extends. Hook (506) is secured into tissue (508), thereby securing implant (502) to the tissue. As shown in FIG. 5A, implant (502) initially is in a curved configuration as it exits delivery sheath (500). However, as implant (502) continues to be deployed from delivery sheath (500), implant (502) loses its curved configuration, assuming a straighter shape, as shown in FIG. 5B. After hook (506) has secured into tissue (508), delivery sheath (500) is further withdrawn, thereby exposing an additional length of implant (502) for deployment into the target site. As implant (502) continues to be deployed from delivery sheath (500), support (504) is stretched in the direction of arrow (A1), and a second hook (510) of implant (502) is hooked into a different region of tissue (508). Referring now to FIG. 5C, once implant (502) has been fully deployed from delivery sheath (500), support (504) is released. The release of support (504) causes support (504) to resume its original form, resulting in the formation of folds in a region (512) of tissue (508) between hooks (506) and (510). This method may or may not be repeated (e.g., to deliver multiple implants to various regions of heart tissue), and may be used, for example, to repair a mitral valve annulus by reshaping the annular tissue.

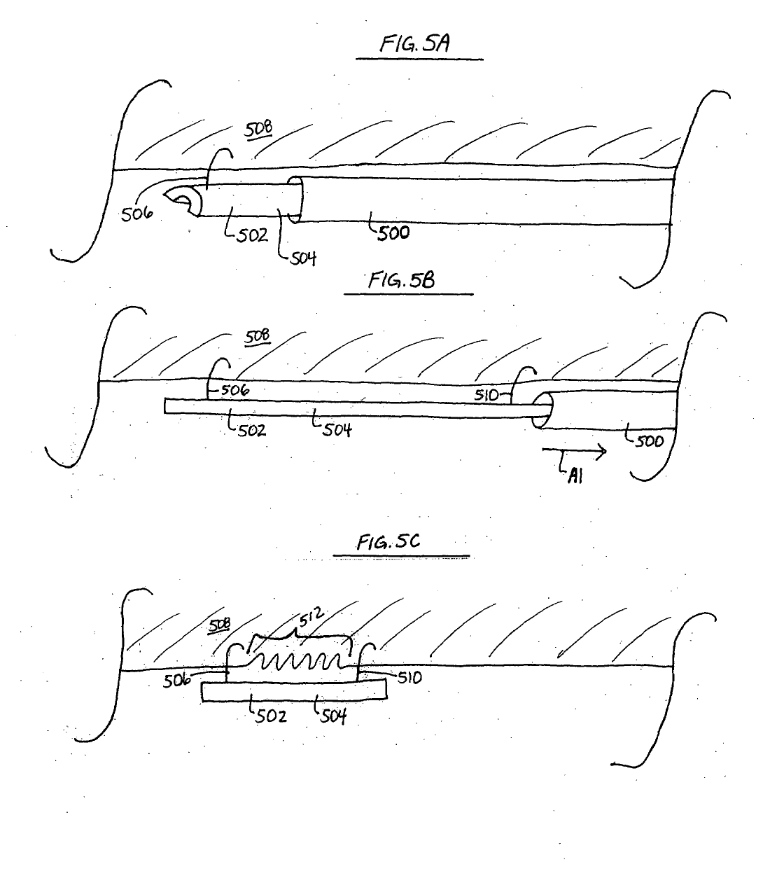

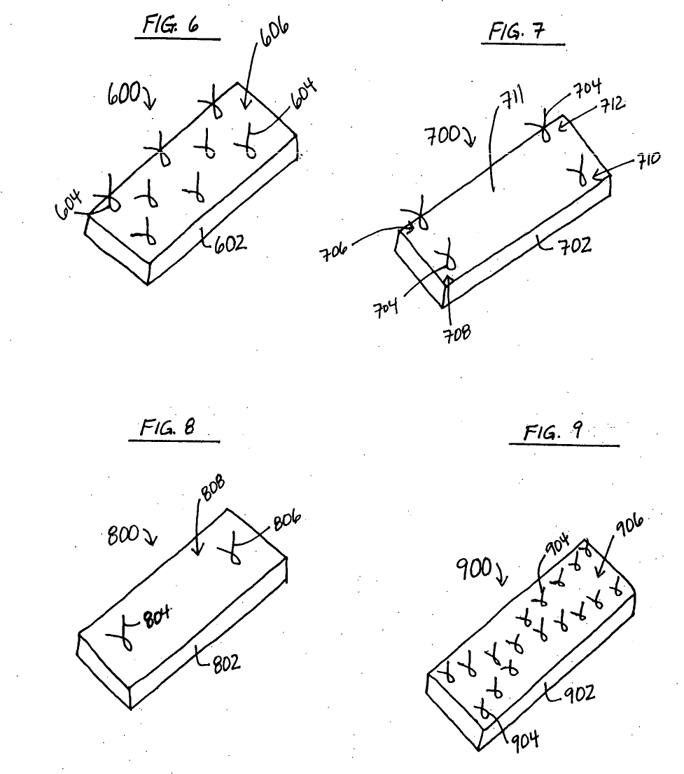

The implants described here may have any suitable configuration of anchor portions. For example, FIGS. 6 and 7 show implants having different arrangements of anchor portions, there shown as looped anchors. More specifically, FIG. 6 shows an implant (600) with a support (602) and anchors (604) spaced relatively uniformly on one side (606) of support (602). In FIG. 7, an implant (700) is shown having a support (702) and anchors (704) extending from each of the corners (706), (708), (710), and (712) of one side (711) of the support.

An implant may include a relatively large number of anchor portions, or may include a relatively low number of anchor portions. For example, the implant (800) shown in FIG. 8 includes a support (802) and only two anchor portions (804) and (806) extending from opposite ends of one side (808) of the support. Implants such as implant (800) may be useful, for example, to temporarily reshape tissue (e.g., by forming one or more folds in the tissue). Temporary reshaping of heart tissue may be used, for example, to determine the appropriate amount of tissue folding to be accomplished during an actual repair procedure. The relatively small number of anchor portions on the implant may allow for easy dislodgment or detachment of the implant from the tissue once the temporary tissue reshaping is no longer needed.

The implants may include different patterns of anchor portions, or may include anchor portions that are arranged on the implant to correspond to the topography of the target tissue. As an example, FIG. 9 shows an implant (900) including a support (902) and multiple anchors (904) extending from one side (906) of the support. Anchors (904) are arranged in an “X” pattern, although other patterns may be used. For example, an implant may include anchor portions that form a curved pattern to correspond to a curve in the mitral valve annulus. Additionally, while implants having a relatively symmetrical arrangement of anchor portions have been shown, some variations of implants may include anchor portions that are asymmetrically arranged. Furthermore, some portions of an implant may include anchor portions that are symmetrically arranged, while other portions of the implant include anchor portions that are asymmetrically arranged.

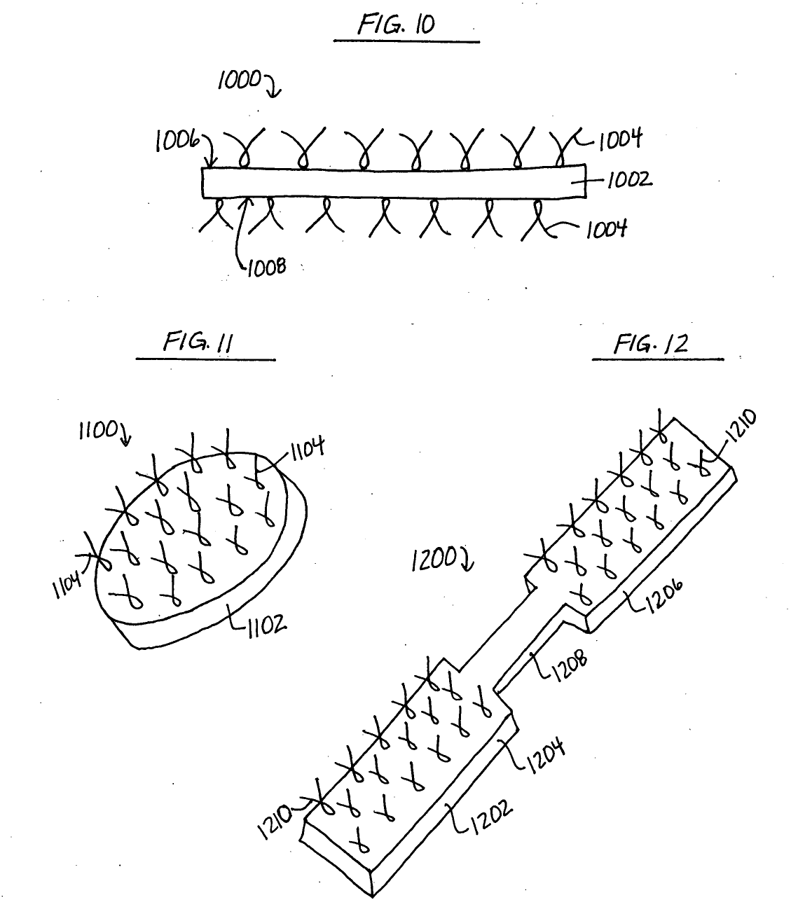

While implants having anchor portions extending from one surface of a support have been shown, certain variations of implants may comprise anchor portions extending from more than one surface of a support. For example, FIG. 10 shows an implant (1000) including a support (1002) and anchors (1004) extending from opposite surfaces (1006) and (1008) of the support. Implant (1000) may be used, for example, in a tissue repair procedure in which the implant is secured into a crevice within the tissue (e.g., a space between a mitral valve leaflet and the ventricular wall). The presence of anchors extending from multiple surfaces of the implant may help the implant better secure into the surrounding tissue. In some variations, the entire surface of the implant may be substantially covered with anchor portions.

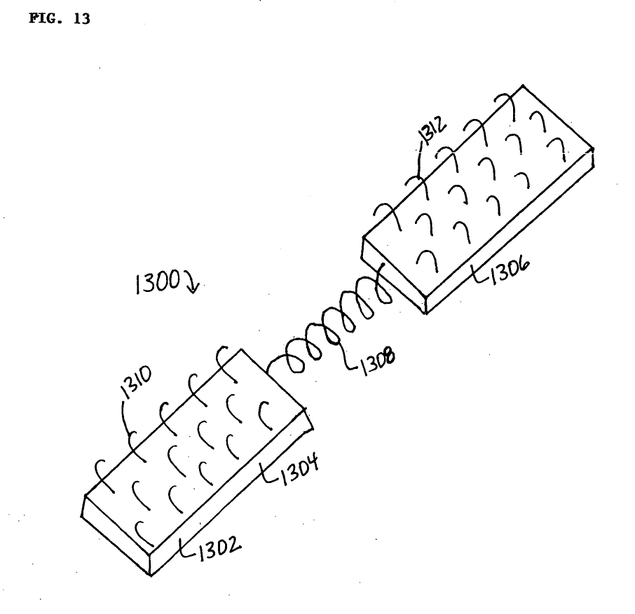

Implants may also include supports having different shapes. As an example, FIG. 11 shows an implant (1100) including a circular support (1102) and anchors (1104) extending from the support. While a circular support is shown, supports may have any of a number of different shapes, such as oval, rectangular, square, triangular, hexagonal, etc., or an irregular shape. For example, FIG. 12 shows an implant (1200) including a support (1202) having a first portion (1204) and a second portion (1206), where the first and second portions are joined by a third portion (1208). In this variation, anchors (1210) extend only from the first and second portions. However, implants including supports having multiple portions may have anchor portions extending from all of the portions, only some of the portions, or just one portion. First, second, and third portions (1204), (1206), and (1208) may all be formed from the same material, or may be formed from different materials. In some variations, the third portion of the support may be formed from a material that is capable of being tensioned or stretched and returned to its original shape once it is no longer under tension. As described above, this stretch and release process may be used to reshape tissue during a tissue repair procedure.

For example, FIG. 13 shows an implant (1300) including a support (1302) having two different portions (1304) and (1306). The different support portions are attached to each other by a spring (1308). In this variation, anchor portions (1310) and (1312) extend from both portions of the support, but the hooks of anchor portions (1310) extending from portion (1304) face in one direction, while the hooks of anchor portions (1312) extending from portion (1306) face in the opposite direction. Some methods of using implant (1300) may include hooking anchor portions (1310) into one region of tissue, stretching spring (1308), and then hooking anchor portions (1312) into another region of tissue. When the implant is released, the spring may resume its original position, thereby causing the different portions of support (1302) to come closer together and form one or more folds in the tissue therebetween. While FIG. 13 shows anchor portions with hooks that face in opposite directions, it should be understood that an implant may include anchor portions with hooks having any of a number of different orientations relative to each other.

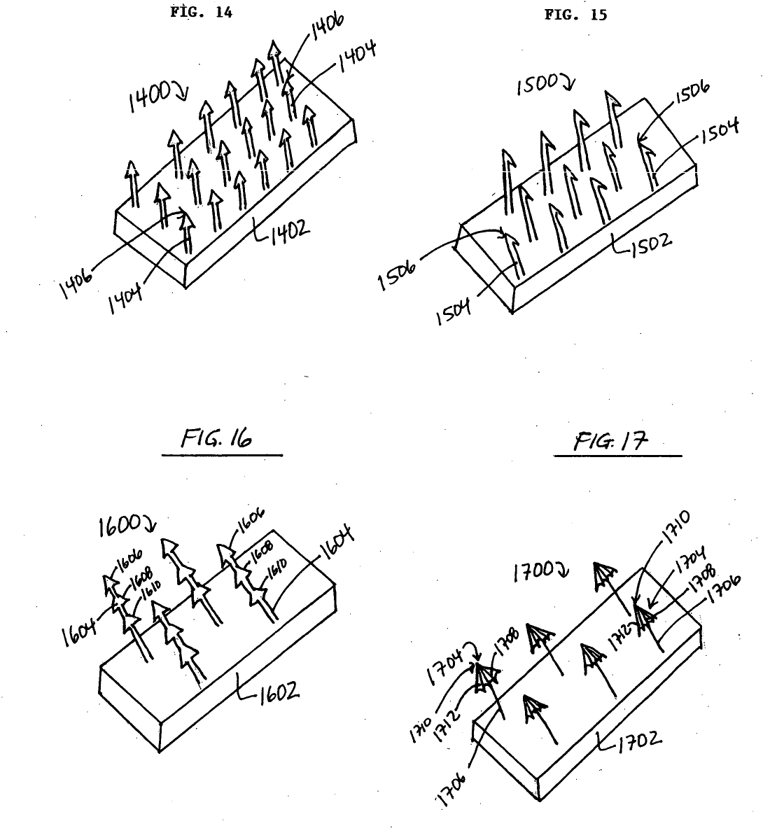

While certain shapes of anchor portions have been shown, it should be understood that the implants described here may have anchor portions having any number of different shapes, sizes, or configurations, as described above. For example, implants may include anchor portions that all have the same shape, that all have different shapes, or some combination of the two. Examples of suitable anchor portions are shown in FIGS. 14-17.

FIG. 14 shows an implant (1400) comprising a support (1402) and barbed anchor portions (1404) extending from the support. The distal ends (1406) of barbed anchor portions (1404) have an arrow configuration. This arrow configuration may enhance the ability of the barbed anchor portions to secure into tissue, and may reduce the likelihood that the barbed anchor portions will become unsecured from the tissue.

FIG. 15 shows an implant (1500), which comprises a support (1502) and barbed anchor portions (1504) extending from the support. The barbed anchor portions (1504) shown here include distal ends (1506) having a harpoon configuration. Barbed anchor portions (1504) may also have a reduced likelihood of becoming unsecured from tissue.

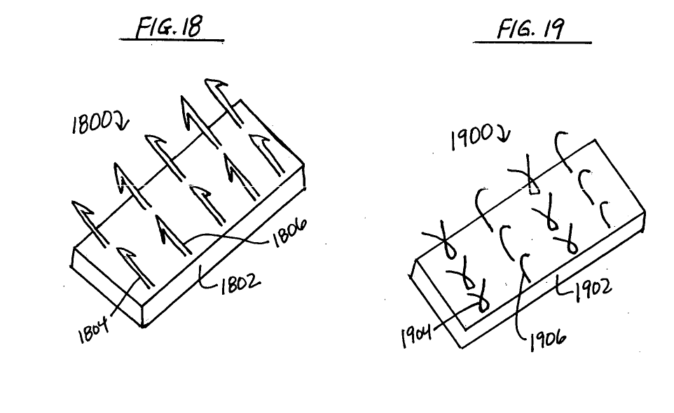

The barbed anchor portions may include one barb or multiple barbs. For example, FIG. 16 shows an implant (1600) having a support (1602) and barbed anchor portions (1604) extending from the support (1602). Barbed anchor portions (1604) each have three barbs (1606), (1608), and (1610). As the number of barbs on an anchor portion increases, the likelihood that the anchor portion will unsecure from tissue may decrease. While implants including barbed anchor portions having the same number of barbs have been described, some variations of implants may include anchor portions having different numbers of barbs. Furthermore, certain variations of implants may include anchor portions with distal ends having different configurations.

FIG. 17 shows an implant (1700) including a support (1702) and multiple umbrella-shaped anchor portions (1704) extending from the support. The umbrella-shaped anchor portions each include a shank (1706), multiple struts (1708) extending from a distal end (1710) of the shank, and a membrane (1712) covering the struts. The struts may be reconfigurable from a reduced configuration, in which the struts are compressed against shank (1706). Upon expansion, the struts resume their umbrella-shaped profile shown in FIG. 17. Thus, anchor portions (1704) may be deployed into tissue in a reduced configuration, and then expanded after entering the tissue. While the umbrella-shaped anchor portions of FIG. 17 include a membrane (1712), in some variations, an anchor portion may include a shank and struts, but no membrane.

As described above, implants may also include anchor portions that face in different directions, and/or may include multiple different types of anchor portions. For example, FIG. 18 shows an implant (1800) including a support (1802) and barbed anchor portions (1804) and (1806) extending from the support. Barbed anchor portions (1804) face in a different direction from barbed anchor portions (1806). FIG. 19 shows an implant (1900) including a support (1902), and anchors (1904) and hooks (1906) extending from support (1902). While implant (1900) as shown comprises only two different types of anchor portions, implants can comprise any number of different types of anchor portions. For example, they may have three, four, five, or ten different types of anchor portions.

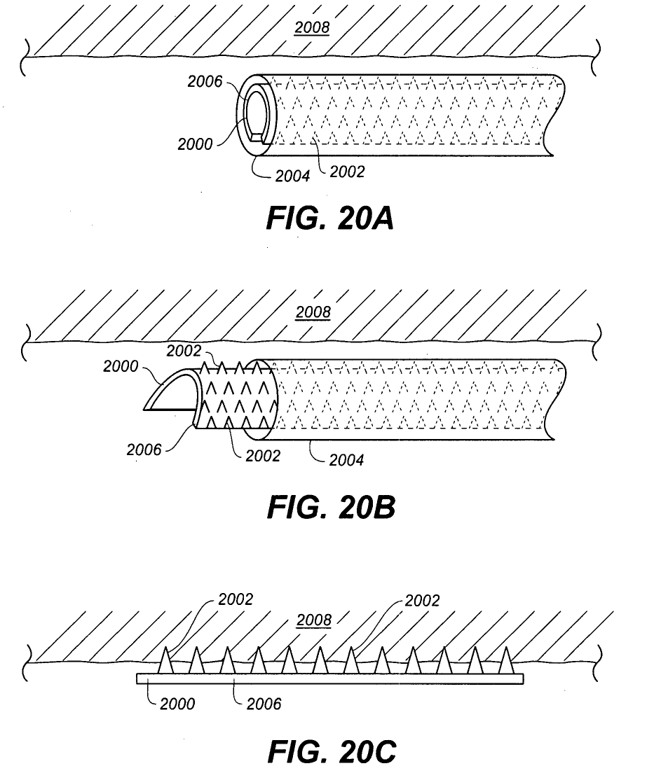

In some variations of implants, the anchor portions may be in the form of nodular or spike-shaped protrusions. For example, FIGS. 20A-20C illustrate the delivery of an implant (2000) having spike-shaped anchor portions (2002) to a target tissue (2008). As shown in FIG. 20A, implant (2000) is partially rolled within a delivery sheath (2004) for advancement to a target tissue site. Implant (2000) includes a support (2006) from which spike-shaped implants (2002) extend. As implant (2000) is deployed from delivery sheath (2004) (e.g., by proximally withdrawing the delivery sheath and pushing the implant out of the delivery sheath with a pusher), implant (2000) unrolls. As a result, spike-shaped anchor portions (2002) face the target tissue, as shown in FIG. 20B. As shown in FIG. 20C, after implant (2000) has been fully deployed, spike-shaped anchor portions (2002) are secured into tissue (2008).

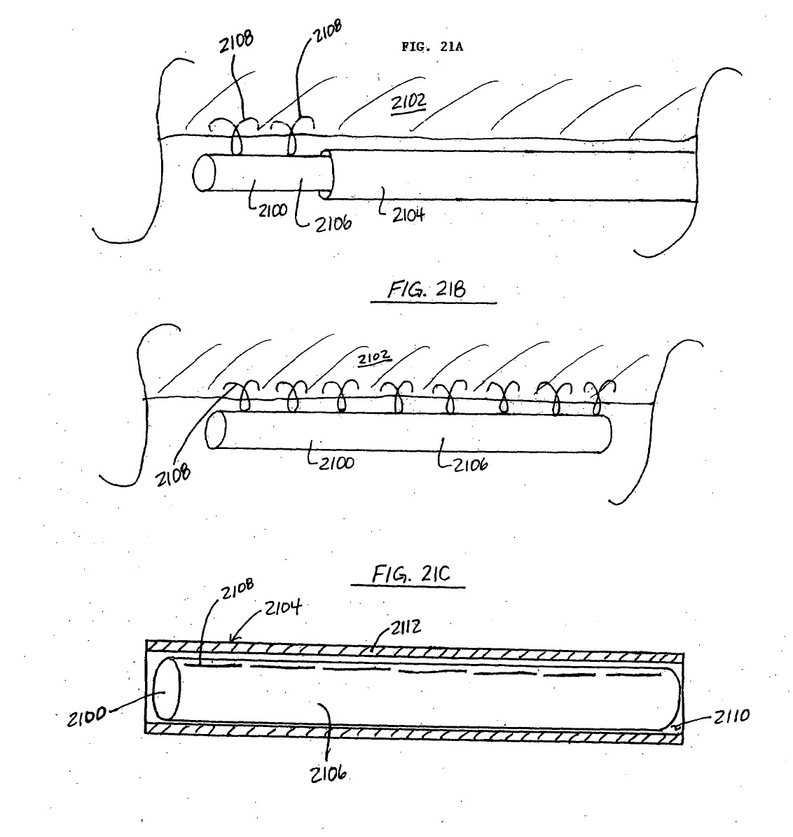

The supports may also be of any suitable configuration, and may not necessarily have definable sides, such as the supports described above. As an example, FIGS. 21A and 21B illustrate the delivery of an implant to a target tissue, where the implant includes a generally cylindrical support.

FIG. 21A shows the delivery of implant (2100) to target tissue (2102) from a delivery sheath (2104) where the implant (2100) comprises a generally cylindrical support (2106) from which self-securing anchors (2108) extend. As implant (2100) is delivered from delivery sheath (2104), self-securing anchors (2108) self-expand and self-secure into tissue (2102). FIG. 21C shows implant (2100) within the lumen (2110) of delivery sheath (2104). As shown in FIG. 21C, anchors (2108) are essentially flattened against generally cylindrical support (2106) by a side wall (2112) of the delivery sheath. However, as implant (2100) is deployed from the delivery sheath, the anchors self-expand and resume their original shape, with their ends curving inward toward each other to form loops. As the anchors self-expand, they also self-secure into the tissue.

While generally cylindrical support (2106) of implant (2100) does not have any lumens, some variations of implants may comprise generally cylindrical supports having one or more lumens. The lumens may be used, for example, to deliver one or more therapeutic agents to a target tissue. In some variations, generally cylindrical supports including lumens may be formed of one or more shape-memory materials. When the implants are deployed into heart tissue (e.g., by withdrawing a sheath), the generally cylindrical supports may expand. This expansion can cause the anchor portions that extend from the generally cylindrical supports to contact and secure into the heart tissue.

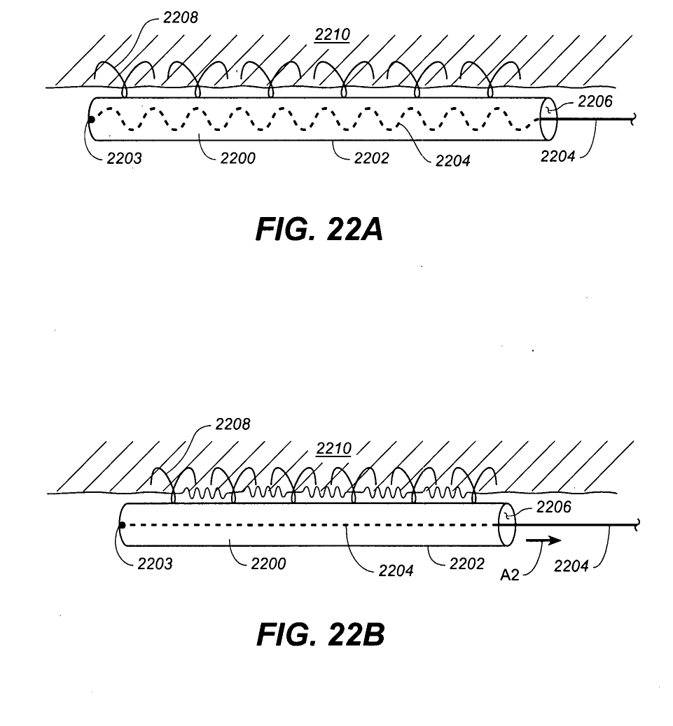

In some variations, the implants comprise tethers. For example, FIG. 22A shows an implant (2200) including a generally tubular support (2202) having a closed end (2203), a tether (2204) loosely disposed within a lumen (2206) of the generally tubular support and attached to closed end (2203), and multiple anchors (2208) extending from generally tubular support (2202) and secured into tissue (2210). As shown in FIG. 22B, when tether (2204) is pulled in the direction of arrow (A2), a cinching effect is achieved, such that folds are formed in the areas of tissue (2210) between adjacent anchors (2208).

The tether may be made from any suitable or desirable biocompatible material. The tether may be braided or not braided, woven or not woven, reinforced or impregnated with additional materials, or may be made of a single material or a combination of materials. For example, the tether may be made from a suture material (e.g., natural fibers, such as silk, and artificial fibers such as polypropylene, polyester, polyester impregnated with polytetrafluoroethylene, nylon, etc.), may be made from a metal alloy (e.g., stainless steel), may be made from a shape memory material, such as a shape memory alloy (e.g., a nickel titanium alloy), may be made from combinations thereof, or may be made from any other suitable biocompatible material.

The tether can be terminated after the desired extent of reduction has been achieved (e.g., as determined by ultrasound and fluoroscopy). As an example, in some variations, the tether can be attached to the proximal-most anchor portion of the implant while the tether is still under tension. The tether can be attached to the proximal-most anchor portion using, for example, one or more adhesives, and/or one or more knotting, crimping, and/or tying techniques. This attachment to the proximal-most anchor portion allows the tension in the tether to be maintained, even when the tether is no longer being pulled proximally. After the tether has been attached to the proximal-most anchor portion, any additional unused length of the tether can be cut proximal to the proximal-most anchor portion, and removed. In some variations, attachment and/or cutting of the tether can be achieved using a termination device, such as a termination catheter. For example, one or more cutting and/or locking catheters can be used to maintain the tension in a tether and to remove the unused portion of the tether, after the desired cinching effect has been achieved by pulling on the tether. As an example, the excess length of the tether may be threaded into a cutting catheter. The cutting catheter can include one or more cutting tools (e.g., blades) that can be used to sever the tether. Termination devices are described, for example, in U.S. patent application Ser. Nos. 11/232,190 and 11/270,034, both of which are hereby incorporated by reference in their entirety.

While certain variations of implants have been described, other variations of implants may be used in tissue repair procedures.

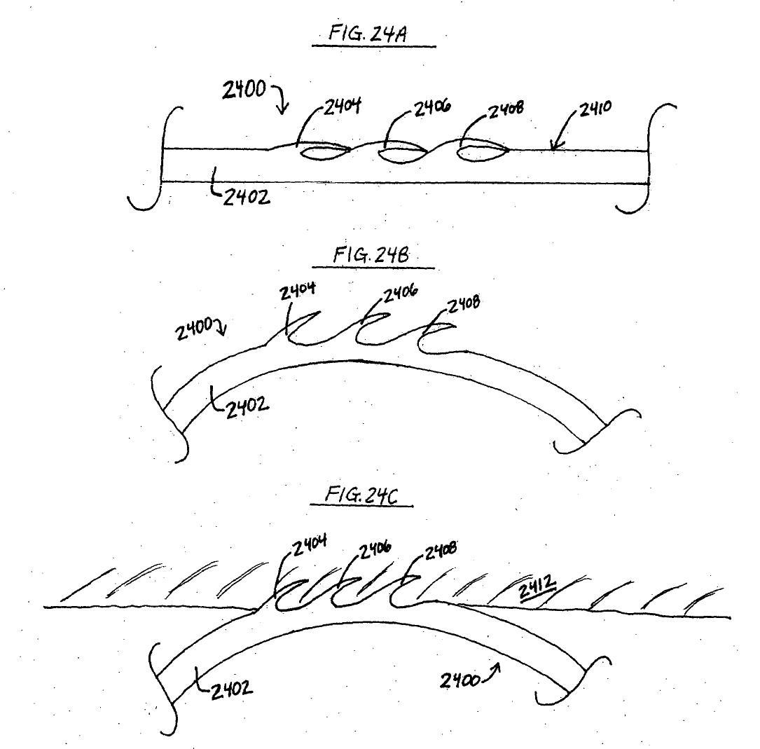

As an example, FIG. 24A shows an implant (2400) including a generally cylindrical support (2402) and anchor portions (2404), (2406), and (2408) extending from, and integrally formed with, the generally cylindrical support. Implant (2400) may be formed, for example, by cutting the anchor portions out of a polymer tube, such as a PEEK tube. As shown in FIG. 24A, anchor portions (2404), (2406), and (2408) are positioned such that they are almost flush with the surface (2410) of generally cylindrical support (2402). The anchor portions may be in this position when, for example, implant (2400) is disposed within a delivery sheath that restrains the anchor portions. As shown in FIG. 24B, when generally cylindrical support (2402) is bent, anchor portions (2404), (2406), and (2408) protrude from the generally cylindrical support, allowing them to be hooked into a target tissue (2412), as shown in FIG. 24C.

While implant (2400) is shown as including a generally cylindrical support, other implants having a similar configuration may include a non-cylindrical support. Furthermore, some variations of implants may include anchor portions that are attached to a generally cylindrical support, either in addition to, or as an alternative to, anchor portions that are integrally formed with a generally cylindrical support.

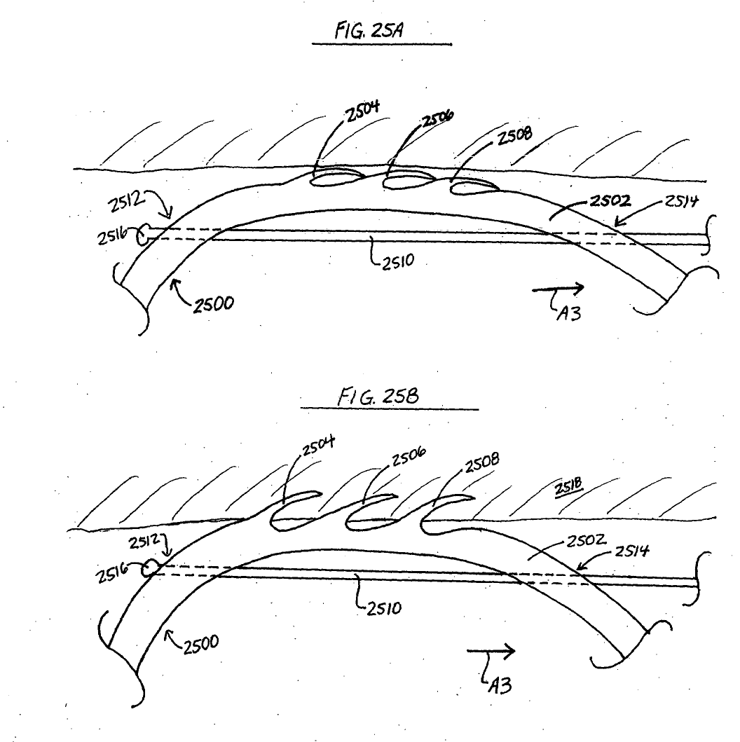

Another variation of an implant is illustrated in FIG. 25A. As shown in FIG. 25A, an implant (2500) includes a generally cylindrical support (2502) and anchor portions (2504), (2506), and (2508) extending from the generally cylindrical support. A rod (2510) extends through two portions (2512) and (2514) of the generally cylindrical support, and can be pulled in the direction of arrow (A3) to bring portions (2512) and (2514) closer together. Rod (2510) may be formed of, for example, one or more metals, metal alloys, and/or polymers. Rod (2510) includes a stop (2516) at one of its ends that may prevent rod (2510) from being pulled through portions (2512) and (2514). As shown in FIG. 25B, when rod (2510) is pulled in the direction of arrow (A3), the protrusion of anchor portions (2504), (2506), and (2508) from generally cylindrical support (2502) is enhanced, allowing the anchor portions to be hooked into a target tissue (2518).

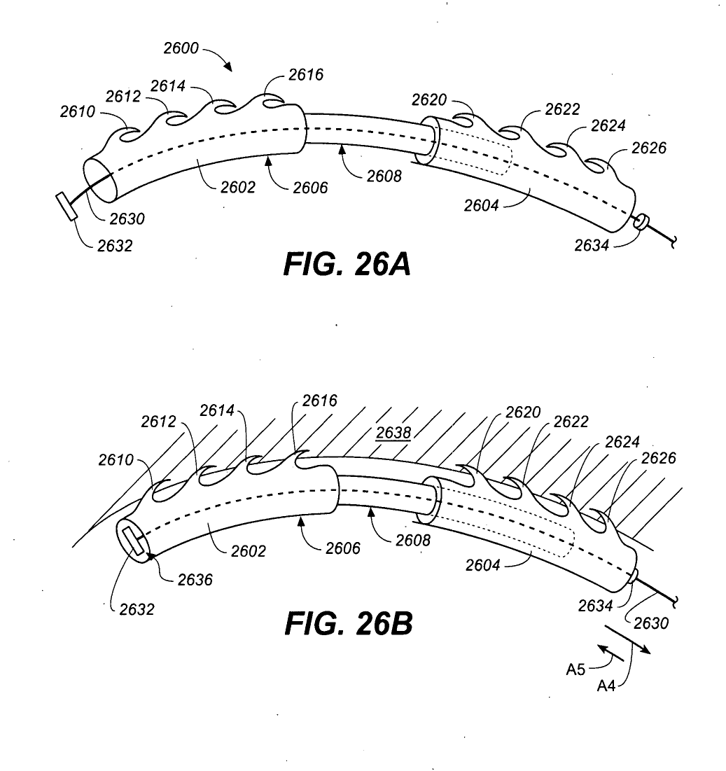

An additional example of an implant is shown in FIG. 26A. As shown in FIG. 26A, an implant (2600) includes a first generally cylindrical support (2602) and a second generally cylindrical support (2604). The first generally cylindrical support includes a larger diameter section (2606) and a smaller diameter section (2608) to which the second generally cylindrical support is telescopically connected. Anchor portions (2610), (2612), (2614), and (2616) extend from first generally cylindrical support (2602), and anchor portions (2620), (2622), (2624), and (2626) extend from second generally cylindrical support (2604). A tether (2630) that is connected to a stop (2632) at one end is threaded through first and second generally cylindrical supports (2602) and (2604). A cinching lock (2634) is movably engaged with the tether at the tether's other end.

Referring to FIG. 26B, during use of implant (2600), tether (2630) may be pulled in the direction of arrow (A4), thereby causing stop (2632) to press against an end (2636) of first generally cylindrical support (2602). Continued pulling on tether (2630) in the direction of arrow (A4) can cause smaller diameter section (2608) to slide further into second generally cylindrical support (2604), while also enhancing the protrusion of the anchor portions from the first and second generally cylindrical supports. This enhanced protrusion may allow implant (2600) to be relatively easily engaged with a target tissue (2638). Furthermore, pulling on the tether may also provide a cinching effect. As shown in FIG. 26B, when the desired amount of anchor portion protrusion and/or cinching has been achieved, cinching lock (2634) may be moved in the direction of arrow (A5) to help secure tether (2630). In certain variations, after cinching, larger diameter section (2606) and second generally cylindrical support (2604) may be about three centimeters apart from each other.

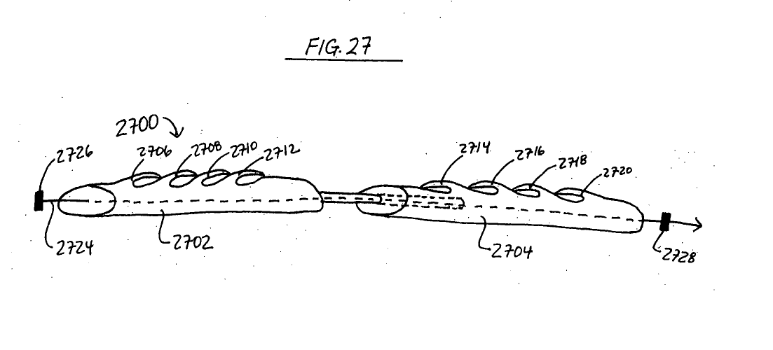

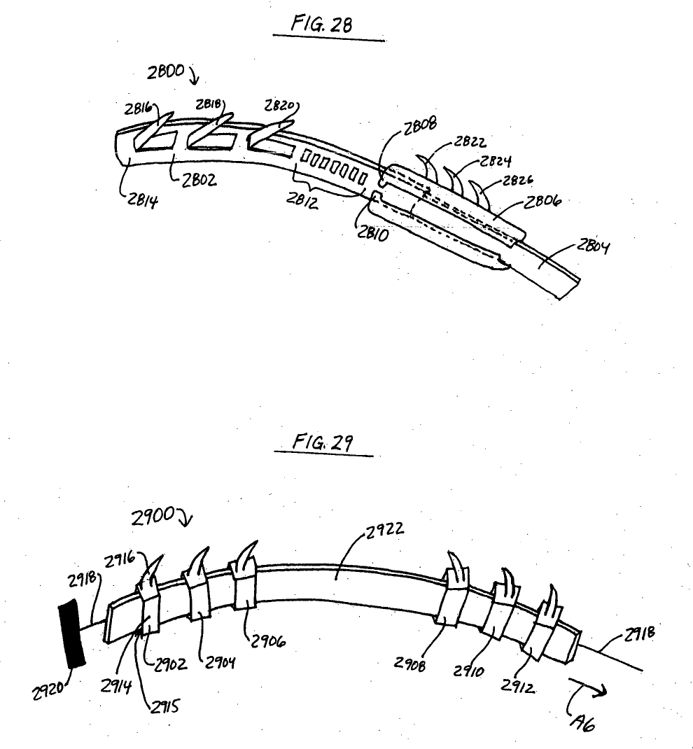

The generally cylindrical supports of implant (2600) are configured such that they have generally circular cross-sections. However, in some variations, an implant may include one or more supports having a non-circular cross-section. For example, FIG. 27 shows an implant (2700) formed of a first support (2702) and a second support (2704). Anchor portions (2706), (2708), (2710), and (2712) extend from first support (2702), and anchor portions (2714), (2716), (2718), and (2720) extend from second support (2704). A tether (2724) is threaded through the first and second supports, and is attached to a stop (2726) at one end. A cinching lock (2728) is movably attached to tether (2724) at the tether's other end. As shown in FIG. 27, first support (2702), as well as second support (2704), both have oval cross-sections. This may make it easier, for example, for a physician to correctly orient the implant during a procedure (e.g., so that the anchor portions are pointing in the desired direction). While oval cross-sections have been described, other cross-sections may be used, such as triangular, irregular, etc. Furthermore, other indicators of orientation may be used. For example, an implant may include a support having a ridge that helps a physician to properly orient the implant at a target site.

While implants having certain configurations have been described, implants having other configurations may be used in a tissue repair procedure. For example, FIG. 28 shows an implant (2800) formed of a strip (2802) that is releasably attached to a holder (2804), and a collar (2806) that is configured to be ratcheted up strip (2802). Collar (2806) includes hooks (2808) and (2810) that are configured to snap into cut-outs (2812) along strip (2802) as collar (2806) is ratcheted up strip (2802). Strip (2802) includes a support (2814) in which the cut-outs are formed, and anchor portions (2816), (2818), and (2820) extending from the support. Additionally, anchor portions (2822), (2824), and (2826) extend from collar (2806). During use of implant (2800), the anchor portions can be hooked into a target tissue, and the collar can be ratcheted up the strip (e.g., using a pusher) to provide a cinching effect. Prior to deployment of implant (2800) at a target site, the anchor portions may be flush or almost flush with the surfaces of strip (2802) and collar (2806). The delivery device that is used to deliver implant (2800) to a target tissue may, for example, include a sheath that temporarily restrains the anchor portions. As the implant is being deployed, the anchor portions may become unrestrained and may protrude to a greater extent from strip (2802) and/or collar (2806). In some variations, a delivery device used for implant (2800) may include a balloon, and implant (2800) may be disposed over the balloon during delivery to a target tissue. When the target tissue has been reached, the balloon may be inflated to enhance the protrusion of the anchor portions from the strip and/or collar. In certain variations, the anchor portions may be connected to a pull-wire that, when pulled upon, can cause the anchor portions to protrude further from the strip and/or collar. These and other devices may be used alone or in combination to enhance the protrusion of the anchor portions on implant (2800) so that the implant may be more easily engaged with a target tissue. In some variations, after the implant has engaged with the target tissue, holder (2804) may be detached from strip (2802), such that only strip (2802) and collar (2806) remain at the target tissue.

Another example of an implant is shown in FIG. 29. As shown there, an implant (2900) includes multiple modules (2902), (2904), (2906), (2908), (2910), and (2912). Each module is formed of a support having an opening, and an anchor portion extending from the support. For example, module (2902) is formed of a support (2914) having an opening (2915), and an anchor portion (2916) extending from support (2914). Implant (2900) further includes a tether (2918) that is attached to a stop (2920) at one end. Tether (2918) is threaded through the openings in the modules, thereby connecting the modules to each other. When implant (2900) is being delivered and deployed to a target tissue, a strip (2922) also is threaded through the openings in the modules, and helps to hold the modules together. In some variations, the strip may include certain features, such as bumps and/or ridges, that enhance the engagement of the strip with the modules. Once implant (2900) has been delivered to a target tissue and one or more of the anchor portions have been hooked into the target tissue, strip (2922) may be removed, or may be allowed to remain in place. Additionally, tether (2918) may be pulled in the direction of arrow (A6) to provide a cinching effect. As shown, stop (2920) is larger than opening (2915) in module (2902), so that tether (2918) may be prevented from inadvertently being pulled all the way through the modules.

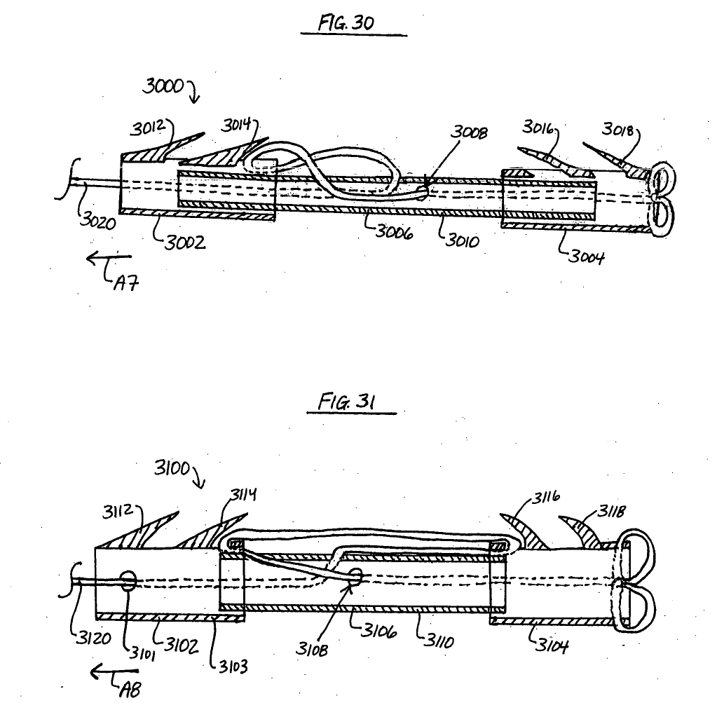

Certain variations of implants may include tethers that are routed such that, when the tethers are pulled upon, they may enhance the engagement of the implants' anchor portions with tissue, and/or provide a relatively good cinching effect. For example, FIG. 30 shows an implant (3000) formed of a first tubular support (3002) and a second tubular support (3004) linked to each other by a tubular member (3006). Tubular member (3006) includes an aperture (3008) in its side wall (3010). Anchor portions (3012) and (3014) extend from first tubular support (3002), and anchor portions (3016) and (3018) extend from second tubular support (3004). A tether (3020) is routed through the first and second tubular supports and the tubular member (with dashed lines indicating that the tether is inside of the implant, and solid lines indicating that the tether is outside of the implant). When the anchor portions are hooked into tissue, and the tether is pulled upon in the direction of arrow (A7), the tether pulls the first and second tubular supports closer together. This, in turn, may result in a cinching effect, and/or may enhance the engagement of the anchor portions with the tissue. Typically, first tubular support (3002) may start to move toward second tubular support (3004) first, with the second tubular support beginning to move toward the first tubular support shortly thereafter. In some variations, after the tether has been pulled to provide the desired amount of cinching, a plug inside of first tubular support (3002) may be used to terminate the tether.

FIG. 31 depicts another variation of an implant including a tether that is specially routed to provide a cinching effect and/or to enhance anchor portion protrusion. As shown in FIG. 31, an implant (3100) includes a first tubular support (3102) having an aperture (3101) in its side wall (3103), and a second tubular support (3104). The first and second tubular supports are connected to each other by a tubular member (3106). Tubular member (3106) includes an aperture (3108) in its outer wall (3110). Anchor portions (3112) and (3114) extend from first tubular support (3102), and anchor portions (3116) and (3118) extend from second tubular support (3104). A tether (3120) is routed through the first and second tubular supports and the tubular member (with dashed lines indicating that the tether is inside of the implant, and solid lines indicating that the tether is outside of the implant). When the anchor portions are hooked into tissue, and the tether is pulled upon in the direction of arrow (A8), the tether pulls the first and second tubular supports toward each other. This, in turn, may result in a cinching effect, and/or may enhance the engagement of the anchor portions with the tissue. Typically, second tubular support (3104) may start to move toward first tubular support (3102) first, with the first tubular support beginning to move toward the second tubular support shortly thereafter. In some variations, after the tether has been pulled to provide the desired amount of cinching, a knot may be pushed down so that it bumps up against aperture (3101), thereby terminating the tether. In certain variations, after the tether has been pulled to provide the desired amount of cinching, the tether may be threaded through a greater extent of first tubular support (3102), and a plug may be pushed into first tubular support (3102) to terminate the tether.

While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the appended claims. For example, while the above-described methods and devices have been described with respect to heart tissue, it should be understood that the above-described methods and devices are contemplated for use with any body tissue that may be accessed percutaneously, through the skin via laparoscopic incision, or through the airways. That is, the detailed description provided here of how these methods and devices are used with respect to the heart anatomy simply represents one illustrative variation of how these methods and devices may be used. For example, the methods may be used with the heart, various vessels, the bladder, the stomach, and the like.

What is claimed is:

1. A method comprising:
securing an implant to a first region of a tissue in the vicinity of a subannular groove of a heart, wherein the implant comprises a support and a first anchor portion extending from a first portion of the support.

2. The method of claim 1, wherein securing the implant to the first region of the tissue comprises securing the first anchor portion into the first region of the tissue.

3. The method of claim 1, wherein securing the implant to the first region of the tissue comprises advancing the implant to the first region of the tissue, and deploying the implant to the first region of the tissue, wherein the first anchor portion self-secures into the first region of the tissue upon deployment of the implant to the first region of the tissue.

4. The method of claim 1, wherein the implant further comprises a second anchor portion extending from a second portion of the support.

5. The method of claim 4, further comprising securing the implant to a second region of heart tissue.

6. The method of claim 5, wherein securing the implant to the second region of heart tissue comprises securing the second anchor portion into the second region of heart tissue.

7. The method of claim 5, wherein securing the implant to the second region of heart tissue comprises deploying the implant to the second region of heart tissue, wherein the second anchor portion self-secures into the second region of heart tissue upon deployment of the implant to the second region of heart tissue.

8. The method of claim 5, further comprising tensioning the support prior to securing the implant to the second region of heart tissue.

9. The method of claim 5, wherein the implant further comprises a tether.

10. The method of claim 9, wherein the heart tissue comprises mitral valve tissue comprising an annulus.

11. The method of claim 10, further comprising pulling the tether proximally to reduce the circumference of the mitral valve annulus.

12. The method of claim 9, wherein the tether comprises a suture.

13. The method of claim 12, wherein the suture comprises polyester impregnated with polytetrafluoroethylene.

14. The method of claim 9, further comprising pulling the tether proximally to create at least one heart tissue fold between the first and second anchor portions.

15. The method of claim 9, wherein the tether is attached to the support.

16. The method of claim 15, wherein the tether has a first end portion that is attached to the support and a second end portion that is not attached to the support.

17. The method of claim 4, wherein at least one of the first and second anchor portions is integrally formed with the support.

18. The method of claim 4, wherein at least one of the first and second anchor portions is attached to the support.

19. The method of claim 18, wherein at least one of the first and second anchor portions is partially embedded in the support.

20. The method of claim 4, wherein the first anchor portion has a different shape from the second anchor portion.

21. The method of claim 4, wherein the first anchor portion has a different size from the second anchor portion.

22. The method of claim 4, wherein the first and second portions of the support are connected by a spring.

23. The method of claim 4, wherein the first and second portions of the support are connected by a third portion of the support.

24. The method of claim 23, wherein the third portion of the support is integrally formed with at least one of the first and second portions of the support.

25. The method of claim 1, wherein the first anchor portion is selected from the group consisting of T-tags, rivets, staples, hooks, and clips.

26. The method of claim 1, wherein the support comprises a polymer.

27. The method of claim 1, wherein the support comprises a metal.

28. The method of claim 1, wherein the support comprises a metal alloy.

29. The method of claim 1, wherein the support comprises a first material, and the first anchor portion comprises a second material that is different from the first material.

30. The method of claim 1, wherein the support and the first anchor portion are formed of the same material.

31. The method of claim 1, wherein the support is in the form of a generally cylindrical member.

32. The method of claim 31, wherein the generally cylindrical member includes at least one lumen.

33. The method of claim 1, wherein the support has a first surface and a second surface opposite the first surface.

34. The method of claim 33, wherein the first anchor portion extends from the first surface, and the implant further comprises a second anchor portion extending from the second surface.

35. The method of claim 1, wherein the implant comprises a plurality of hooks.

36. The method of claim 1, wherein the implant comprises a plurality of anchor portions that are configured to secure to heart tissue.

37. The method of claim 36, wherein the plurality of anchor portions are configured to secure to heart tissue such that disengagement of at least one of the anchor portions from the heart tissue does not result in disengagement of the implant from the heart tissue.

38. A method comprising:
advancing a catheter to a first region of a tissue in the vicinity of a subannular groove of a heart; and
deploying an implant from the catheter to the first region of tissue, wherein the implant comprises a support and a first anchor portion extending from a first portion of the support.

39. The method of claim 38, wherein the implant further comprises a second anchor portion extending from a second portion of the support.

40. The method of claim 38, wherein the catheter comprises a lumen, and prior to deployment of the implant to the first region of tissue, the implant is at least partially disposed within the lumen of the catheter.

41. The method of claim 40, wherein prior to deployment of the implant to the first region of tissue, the implant is in a rolled or folded configuration within the lumen of the catheter.

42. The method of claim 41, wherein the implant is configured to unroll or unfold when the implant is deployed from the catheter.

43. The method of claim 38, wherein deploying the implant from the catheter comprises using a pusher to advance the implant through the catheter.

44. The method of claim 38, wherein the catheter comprises an expandable member.

45. The method of claim 44, wherein prior to deployment of the implant to the first region of tissue, the implant is supported by the expandable member.

46. The method of claim 45, wherein deploying the implant from the catheter to the first region of tissue comprises expanding the expandable member.

47. The method of claim 45, wherein prior to deployment of the implant to the first region of tissue, the implant is disposed between a sheath and the expandable member.

48. The method of claim 47, wherein deploying the implant from the catheter to the first region of tissue comprises withdrawing the sheath.

49. The method of claim 44, wherein the expandable member comprises a balloon.

50. The method of claim 44, wherein the expandable member comprises a shape-memory material.

51. An implant comprising:
a support; and
a plurality of anchor portions extending from the support,
wherein the plurality of anchor portions comprise at least three anchor portions having a non-linear relationship with respect to each other, and the implant is configured to secure to a first region of mitral valve tissue.

52. The implant of claim 51, further comprising a tether.

53. The implant of claim 52, wherein the mitral valve tissue comprises an annulus, and the tether is configured to be pulled proximally to reduce the circumference of the mitral valve annulus.

54. The implant of claim 51, wherein at least one of the anchor portions is integrally formed with the support.

55. The implant of claim 51, wherein at least one of the anchor portions is selected from the group consisting of T-tags, rivets, staples, hooks, and clips.

56. The implant of claim 51, wherein the support comprises a polymer.

57. The implant of claim 51, wherein the support has a first surface and a second surface that is different from the first surface.

58. The implant of claim 57, wherein the second surface is opposite the first surface.

59. The implant of claim 57, wherein the implant comprises a first anchor portion extending from the first surface, and a second anchor portion extending from the second surface.

60. The implant of claim 51, wherein the plurality of anchor portions comprise a plurality of hooks.

61. The implant of claim 51, wherein the plurality of anchor portions are configured to secure into heart tissue such that disengagement of at least one of the anchor portions from the heart tissue does not result in disengagement of the implant from the heart tissue.

62. An implant comprising:
a support; and
a plurality of anchor portions extending from the support,
wherein the plurality of anchor portions form a non-linear array on the support, and the implant is configured to secure to a first region of mitral valve tissue.

63. The implant of claim 62, wherein the plurality of anchor portions comprise a plurality of hooks.

64. The implant of claim 62, wherein the plurality of anchor portions are configured to secure into heart tissue such that disengagement of at least one of the anchor portions from the heart tissue does not result in disengagement of the implant from the heart tissue.
